(19) United States
(12) Patent Application Publication
(10) Pub. No.: US 2018/0235536 A1
(43) Pub. Date: Aug. 23, 2018

(54) ASSESSMENT APPARATUS FOR PAIN MEASUREMENT

(71) Applicant: Key Clinical Trial Services LLC, Salt Lake City, UT (US)

(72) Inventor: Georgia SIMMONS, Salt Lake City, UT (US)

(21) Appl. No.: 15/900,214

(22) Filed: Feb. 20, 2018

Related U.S. Application Data
(60) Provisional application No. 62/461,513, filed on Feb. 21, 2017.

(51) Int. Cl. A61B 5/00

(52) U.S. Cl. CPC A61B 5/4827; A61B 5/684; A61B 5/6831; A61B 5/0053

(57) ABSTRACT

An armband for measuring pain during medical studies including a strap portion configured to be secured around a person's upper arm, an opening in the strap portion configured to secure a stimulus in place against the person's upper arm, and at least one marker configured to be secured to the strap portion to mark a designated spot on the strap portion

### CROSS-REFERENCE TO RELATED APPLICATIONS

The present application claims priority to U.S. Patent Application No. 62/461,513, filed Feb. 21, 2017 and entitled “Assessment Apparatus for Measuring Pain.”

### BACKGROUND OF THE INVENTION

The present invention relates to the ability to measure pain in the Delayed Onset Muscle Soreness (“DOMS”) pain model for studies of new drugs. In such studies, participants complete a desired bicep muscle stressing regimen. After a period of 24-48 hours, soreness develops in the muscle. Participants return to the study center for administration of the drug under investigation. Participants perform a series of exercises designed aggravate pain and rate how their muscle feels before and after receiving the drug under investigation. Delayed Onset Muscle Soreness is vague and difficult for participants to quantify. Participants have been at risk for injury due to the level of exercise required to induce sufficient pain for assessment. Therefore, a need exists for a device to improve the participants ability to identify and quantify pain in connection with these studies.

### BRIEF SUMMARY OF THE INVENTION

The present invention relates to an armband for measuring pain during medical studies including a strap portion configured to be secured around a person's upper arm, an opening in the strap portion configured to secure a stimulus in place against the person's upper arm, and at least one marker configured to be secured to the strap portion to mark a predetermined spot on the strap portion.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a front perspective view of an armband for measuring pain in accordance with a preferred embodiment of the present invention.

### DETAILED DESCRIPTION OF THE INVENTION

Certain terminology is used in the following description for convenience only and is not limiting. Unless specifically set forth herein, the terms “a”, “an” and “the” are not limited to one element but instead should be read as meaning “at least one”. The words “right,” “left,” “lower,” and “upper” designate directions in the drawings to which reference is made. The words “inwardly” or “distally” and “outwardly” or “proximally” refer to directions toward and away from, respectively, the geometric center or orientation of the device and related parts thereof. The terminology includes the above-listed words, derivatives thereof and words of similar import.

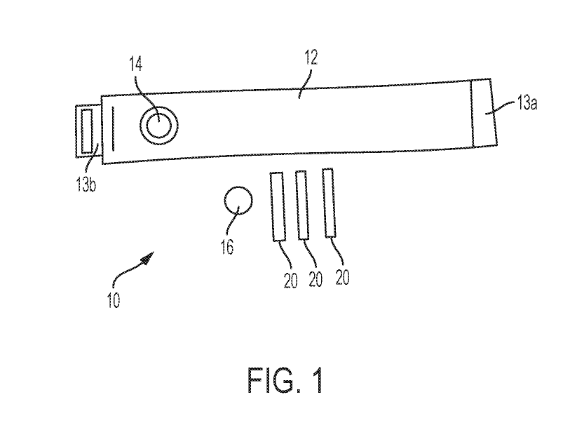

Referring to FIG. 1, a preferred embodiment of an armband 10 is disclosed for measuring pain during medical studies. The armband 10 includes a strap portion 12 configured to be secured around a person's upper arm, which may also take the form of a sleeve or any other form suitable for wrapping around a body part. The strap portion 12 is preferably a wide elastic strip with a Velcro® tab 13a located at one end thereof, but is not so limited. Preferably, a slide 13b is located at an opposed end of the strap portion 12. The slide 13b preferably includes a piece of plastic or metal with a slot formed therethrough, although other forms can be used as well. The armband is preferably secured by inserting the Velcro® tab 13a through the slide 13b, followed by fastening the Velcro® tab 13a to itself or some other portion of the strap portion 12. The strap portion 12 further includes an opening 14 configured to secure a stimulus 16 in place against the person's upper arm. Alternately, the opening 14 may take the form of a depression or any other element suitable for holding an object in place. The opening 14 is preferably a round grommet or metal ring, but is not so limited. The stimulus 16 is preferably a ball, but is not so limited. The armband 10 also preferably includes at least one marker 20 configured to be secured to the strap portion 12 to mark a predetermined spot on the strap portion 12. The markers 20 are preferably affixed to the strap portion 12 to mark the tightness of the strap portion 12 on the wearer's arm, so that the strap portion 12 can be detached from the arm and reapplied at the same tightness. The markers 20 are preferably thin colored strips with a piece of Velcro® on one side for attaching to the strap portion 12, but may alternately take on any shape or form capable of indicating a location on the strap portion 12 or person wearing the strap portion 12.

In use, the strap portion 12 is secured around a study participant's upper arm and bicep. The participant situates the strap portion 12 such that the opening 14 is located directly over the most painful portion of the participant's arm. A study staff member then uses a pen or similar tool (not shown) to mark the participant's arm through the opening 14. The strap portion 12 is then removed from the participant's arm so that it can be reapplied with the stimulus 16 secured between the opening 14 and the participant's arm. The strap portion 12 is then marked with the marker 20 to denote the tightness at which it is applied. The participant can then perform the movement designed to incite pain.

It will be appreciated by those skilled in the art that changes could be made to the embodiment described above without departing from the broad inventive concept thereof. It is understood, therefore, that this invention is not limited to the particular embodiment disclosed, but it is intended to cover modifications within the spirit and scope of the present invention as defined by the present disclosure.

I/We claim:

1. An armband for measuring pain during medical studies, the armband comprising:
a strap portion configured to be secured around a person's upper arm;
an opening in the strap portion configured to secure a stimulus in place against the person's upper arm; and
at least one marker configured to be secured to the strap portion to mark a predetermined spot on the strap portion.

2. The armband of claim 1, wherein the stimulus is a ball.

3. The armband of claim 1, wherein the opening is round.

4. The armband of claim 4, wherein the opening is a grommet or ring.

5. The armband of claim 1, wherein the marker is secured to the strap portion by Velcro®.

6. The armband of claim 1, wherein the strap portion is constructed of an elastic material.

7. The armband of claim 1, wherein a first end of the strap portion is configured to be secured to a second end of the strap portion when secured around a person's upper arm.

8. The armband of claim 7, wherein the first end of the strap portion is threaded through a slide on the second end and secured by Velcro® to itself.

9. A method for measuring pain during medical studies, the method comprising:
placing a strap portion around a person's upper arm such that an opening in the strap portion is located adjacent the most painful portion of the participant's arm;
marking the most painful portion of the participant's arm through the opening;
removing the strap portion from the person's upper arm;
placing a stimulus against the marking and replacing the strap portion around the person's upper arm such that the stimulus is held in place between the most painful portion of the participant's arm and the opening of the strap portion; and
marking the strap portion with a marker to mark the tightness at which the strap portion is applied.

10. The method of claim 9, further comprising moving the participant's arm so as to incite pain.
